(19) United States
(12) Patent Application Publication
(10) Pub. No.: US 2021/0123855 A1
(43) Pub. Date: Apr. 29, 2021

(54) CLASSIFICATION, SCREENING AND DIAGNOSTIC METHODS AND APPARATUS

(71) Applicants: Epigem Limited, Cleveland (GB); University of Zurich, Zurich (CH)

(72) Inventors: Timothy George RYAN, Great Ayton (GB); Asya MAKHRO, Zurich (CH)

(21) Appl. No.: 16/641,031

(22) PCT Filed: Aug. 15, 2018
(86) PCT No.: PCT/EP2018/072127

§ 371 Date: Feb. 21, 2020

(30) Foreign Application Priority Data
Aug. 23, 2017 (GB) 1713568.2

(51) Int. Cl. G01N 15/14; G01N 15/02; G01N 33/49; G06T 7/00

(52) U.S. Cl. CPC G01N 15/1475; G01N 15/0227; G01N 2015/0073; G06T 7/0014; G01N 33/49

(57) ABSTRACT

A method of measuring the morphological profile of a sample of red blood cells including measuring the projected area distribution of the cells and identifying characteristic peak areas from the distribution. The spectrum of peak areas may be compared with those from known blood disorders, and provides a classification, screening and diagnostic methods for a range of blood disorders, especially anaemias. Apparatus for carrying out the methods is also provided, which in some embodiments includes the use of microfluidic devices.

### FIELD OF THE INVENTION

The invention relates to methods and apparatus for monitoring the physiological state of red blood cells (RBCs). This provides methods for detecting abnormalities in such cells, giving the ability to pre-screen patients for such abnormalities (such as those produced by anaemias, especially rare anaemias). Methods are also presented for diagnosing such diseases. Apparatus for carrying out such methods are also disclosed.

Background and Prior Art

It is known that red blood cells can adopt a number of morphological forms, and that the presence of some of these forms can be indicative of a disease state.

Automated blood cell analysers are able to provide counts of red and white blood cells, and of platelets. Centrifugal segregation of red blood cells in a density gradient gives an indication of the presence and relative abundance of the various morphological forms of the red blood cells but requires 0.5-1.0 ml of blood, limiting the use of this technique to adult patients. However, accurate identification of the distribution of morphological forms of RBCs typically requires time-consuming and expensive analysis by direct visual inspection of cells by a skilled technician. Blood films (smears) that are currently used for assessing RBC morphology do not always adequately reflect true morphological appearance of cells in suspension.

Some recent advances (Deb, N. and Chakroborty, S., “A Noble Technique for Detecting Anemia through Classification of Red Blood Cells in a Blood Smear”, IEEE International Conference on Recent Advances and Innovations in Engineering (ICRAIE-2014), May 9, 2011, Jaipur, India. FIG. 3 of that paper shows that a lot of shape artefacts may be introduced by this technique: cells are “angular” and artificially elongated in one direction, also “echinocytes” may appear as a result of film drying. The authors show that analysis of images of RBCs involving extraction of colour data from images, measurement of cell aspect ratios and the calculation of Fourier descriptors for blood cells (involving the calculation of the spatial frequency content of the boundary of the cells) can be used to provide at least a degree of differentiation between different RBC morphologies and differentiation between white blood cells and nucleated RBCs.

Despite these advances, automatic morphological analysis of RBCs for use in diagnostic tests, pre-screening and patient monitoring is not yet available.

For many anaemias, especially the rare anaemias, genetic analysis to identify individual mutations or groups of mutations is required to reach even a putative diagnosis.

It is among the objects of the present invention to attempt a solution to these and other problems.

### SUMMARY OF THE INVENTION

Accordingly, the invention provides a method of measuring the morphological profile of a sample of red blood cells (RBCs) comprising the steps of: (a) measuring the greatest projected area of each RBC in a population of said RBCs to create a projected area distribution of the RBCs; and (b) identifying characteristic peak areas from said projected area distribution;

each relative peak area indicating the proportion of phenotypic cell types within the RBC sample.

Preferably, said peak areas are the areas of those peaks located closest to the following projected areas: Peak 1: 25 μm²; Peak 2: 30 μm²; Peak 3: 36 μm²; Peak 4: 42 μm²; Peak 5: 46 μm²; Peak 6: 53 μm²; Peak 7: 57 μm²; Peak 8: 63 μm², and wherein each peak indicates the following RBC morphology; Peak 1: terminal echinocytosis; Peak 2: echinocytosis; Peak 3: stomato-RBC; Peak 4: stomato-biconcave; Peak 5: tense biconcave; Peak 6: common biconcave; Peak 7: wavy relaxed biconcave; and Peak 8: relaxed biconcave.

The invention also provides a method of pre-screening for a disorder affecting red blood cells (RBCs) in a subject comprising the steps of: (a) providing a sample of red blood cells previously obtained from a subject; (b) measuring the greatest projected area of each RBC in a population of said RBCs to create a projected area distribution of the RBCs; (c) providing a database storing like projected area distributions of RBCs obtained from subjects including those with a range of such disorders; and (d) comparing said measured projected area distribution to those stored in said database to find a closest match, the potential RBC disorder being that associated with the matched distribution.

Preferably said disorder is a red blood cell disorder, and more preferably, an anaemia.

Preferably, said anaemia is an anaemia selected from the group consisting of: Aceruloplasminemia; Adenosine deaminase increased activity (ADA); Adenylate kinase deficiency; Aldolase deficiency; Alpha-thalassaemia—trait or carrier; Aplastic anemia; Atransferrinemia; Atypical hemolytic uremic syndrome; Autoimmune Haemolyitic Anaemia; Beta-thalassaemia—trait or carrier; Beta-thalassaemia major (and intermedia); CDA with thrombocytopenia (GATA I mutation); Compound heterozygous sickling disorders; Congenital acanthocytosis; Congenital dyserythropoietic anaemia type I; Congenital dyserythropoietic anaemia type II; Congenital dyserythropoietic anaemia type III; Delta Beta-thalassaemia; Diamond-Blackfan-Anemia; DMT1-deficiency anaemia; Fanconi anaemia; Gamma-glutamyl-cysteine synthetase deficiency; GLRX5-related Sideroblastic anaemia; Glucose phosphate isomerase deficiency; Glucose-6-phosphate dehydrogenase deficiency; Glutathione reductase deficiency; Glutathione synthetase deficiency; Haemoglobin C disease; Haemoglobin D disease; Haemoglobin E disease; Haemoglobin H disease; Haemoglobin Lepore; Haemoglobin M with anaemia; Hereditary Elliptocytosis; Hereditary persistance of fetal haemoglobin; Hereditary Spherocytosis; Hereditary Stomatocytosis; Hexokinase deficiency; Imerslund-Grasbeck-Syndrome; Iron—refractory iron deficiency Anemia; Kearns-Sayre syndrome; Lecithin cholesterol acyltransferase deficiency; Other thalassaemias; Other types of congenitale dyserythropoetic Anemia; Paroxysmal nocturnal hemoglobinuria; Pearson's Syndrome; Phosphofructokinase deficiency; Phosphoglycerate kinase deficiency; Pyrimidine 5 nucleotidase deficiency; Pyruvate kinase deficiency; Refractory Anaemia (Myelodysplasia); Severe congenital hypochromic anemia with ringed sideroblasts; Sickle cell anaemia; Sickle cell trait; Sideroblastic anaemia associated with ataxia; SLC25A38-related Sideroblastic anaemia; Thiamine-responsive megaloblastic anemia; Triose phosphate isomerase deficiency; Unstable haemoglobin; and X linked sideroblastic anaemia (gene ALAS2).

Said RBC disorder can also be a conditions associated with stress erythropoiesis.

More preferably, said anaemia is selected from the group consisting of: Hereditary Spherocytosis; and Hereditary Xerocytosis.

The invention also provides a method of identifying a subject likely to have a mutation associated with a red blood cell (RBC) disorder comprising the steps of: (a) providing a sample of red blood cells previously obtained from a subject; (b) measuring the greatest projected area of each RBC in a population of said RBCs to create a projected area distribution of the RBCs; (c) providing a database storing like projected area distributions of RBCs obtained from subjects including those with a range of such mutations; and (d) comparing said measured projected area distribution to those stored in said database to find a closest match, the potential mutation being that associated with the matched distribution.

Preferably, said mutation is associated with a gene selected from the group consisting of: ANK1 (coding for erythrocytic Ankyrin 1 protein); SPTB (coding for erythrocytic spectrin beta chain protein); SCL4A1 (coding for erythrocytic spectrin alpha chain protein); and EPB42 (coding for erythrocyte membrane protein band 4.2).

In any such method, it is preferred that the greatest projected area of each RBC is measured by: (a) allowing said RBCs to sediment onto a surface; and (b) measuring the projected surface area of each RBC in a direction substantially normal to the plane of said surface.

Also in any such method, it is preferred that said sample of red blood cells is diluted before measurement, preferably with a diluent containing an albumin.

Also in any such method, it is preferred that said cells are exposed to a stressor before measurement.

Preferably, said stressor is selected from the group consisting of: osmotic stress; pH stress; temperature stress; and mechanical stress.

In any method of the invention it is also preferred that said projected area is measured by superimposing an ellipse over an image of each RBC, said ellipse having major and minor radii r₁ and r₂, and said projected area being measured as π, r₁, r₂.

Also in any such method using a database, it is preferred that said database of like projected area distributions contains averaged projected area distributions taken from a plurality of subjects.

The invention further provides apparatus for carrying out a method according to a method described herein, said apparatus comprising: (a) a chamber for accepting a sample of RBCs; (b) imaging apparatus to image cells within said sedimentation chamber; (c) a processor configured to measure the distribution of greatest projected areas of said cells; (d) a database storing projected area distributions of RBCs obtained from subjects including those with a range of disorders affecting RBC morphology; and (e) a processor configured to compare said measured distribution of projected areas to those stored in said database to find a closest match between said measured and stored projected area distributions.

Preferably, said chamber is a sedimentation chamber, and more preferably said sedimentation chamber comprises a microfluidic chamber.

Along with raw diagnosis, these methods and apparatus may be used for assessment of disease severity for individual patients. Furthermore they may also be used for monitoring of therapeutic efficacy of treatment comparing the changes in projected areas distribution density for individual patient over time.

Among the advantages of the inventions is that the techniques exclude handling- and drying-related deformations of morphology and allows visualisation of shapes even for cells with unstable membrane such as those for rare hereditary hemolytic anemia.

### BRIEF DESCRIPTION OF THE FIGURES

The invention will be described with reference to the accompanying drawings, in which:

FIG. 1 is an average population density distribution of the projected areas of samples of red blood cells taken from healthy subjects;

FIG. 2 illustrates a method of approximating the projected area of a red blood cell;

FIG. 3 shows area population density distributions of RBCs from subjects diagnosed with a Band 3 mutation;

FIG. 4 shows the mean area population density distributions of RBCs from subjects diagnosed with a Band 3 mutation compared to that of healthy volunteers;

FIG. 5 shows area population density distributions of RBCs from subjects diagnosed with an SPTA mutation;

FIG. 6 shows the mean area population density distributions of RBCs from subjects diagnosed with an SPTA mutation compared to that of healthy volunteers;

FIG. 7 shows area population density distributions of RBCs from subjects diagnosed with an SPTB mutation;

FIG. 8 shows the mean area population density distributions of RBCs from subjects diagnosed with as SPTB mutation compared to that of healthy volunteers;

FIG. 9 shows mean area population density distributions of RBCs from subjects diagnosed with a range of pathologies and from a healthy control subject;

FIG. 10 shows a flow diagram of a method of the invention; and

FIGS. 11-14 illustrate, schematically, apparatus for carrying out methods of the invention.

### DESCRIPTION OF PREFERRED EMBODIMENTS

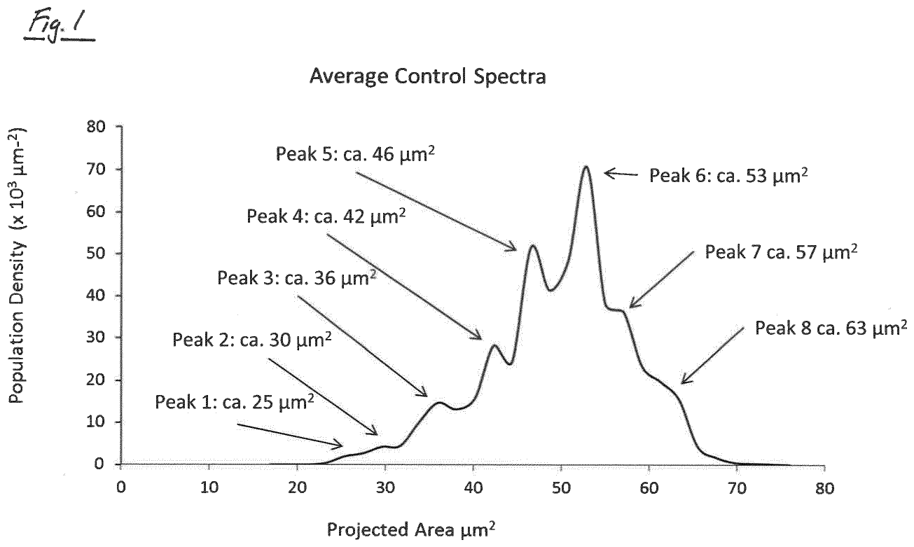

The inventors have surprisingly found that when the projected surface areas of members of a population of red blood cells are measured, the areas do not form a smooth distribution, but are clustered around characteristic peaks. These distributions resemble a projected area spectrum, characteristic of the state of the blood sample and reflect several morphologically defined populations of RBCs corresponding to well-known classes of cells (e.g. discocytes, disco-stomatocytes, stomatocytes, spherocytes, echinocytes). FIG. 1 shows the average projected surface area distribution (or “spectrum”) from eleven samples of blood from healthy subjects. Heparinized blood samples were suspended in a dilution medium at a dilution of 1:1000. The cells were allowed to sediment on a microscope slide, and were imaged using a Zeiss Axiovert 200M microscope with a 100× objective. When the cells sediment, they lie with their largest faces substantially parallel to the plane of the slide (rather than e.g. landing on their edges), and so the image observed is in effect the largest projected surface area of the RBC.

In this experiment, the dilution medium was a blood plasma analogue of the following composition:

| | Concentration |
|---|---|
| Component | (mM) |
| NaCl | 140 |
| KCl | 4 |
| MgCl₂ | 0.75 |
| CaCl2 | 2 |
| ZnCl2 | 0.015 |
| alanine | 0.2 |
| glutamate-Na | 0.2 |
| glycine | 0.2 |
| arginine | 0.1 |
| glutamine | 0.6 |
| glucose | 10 |
| HEPES-imidazole | 20 |

In addition, the medium contained 0.1% (w/v) BSA (bovine serum albumin), and was adjusted to pH 7.4. The procedure was carried out at room temperature (22-27° C., typically 25° C.).

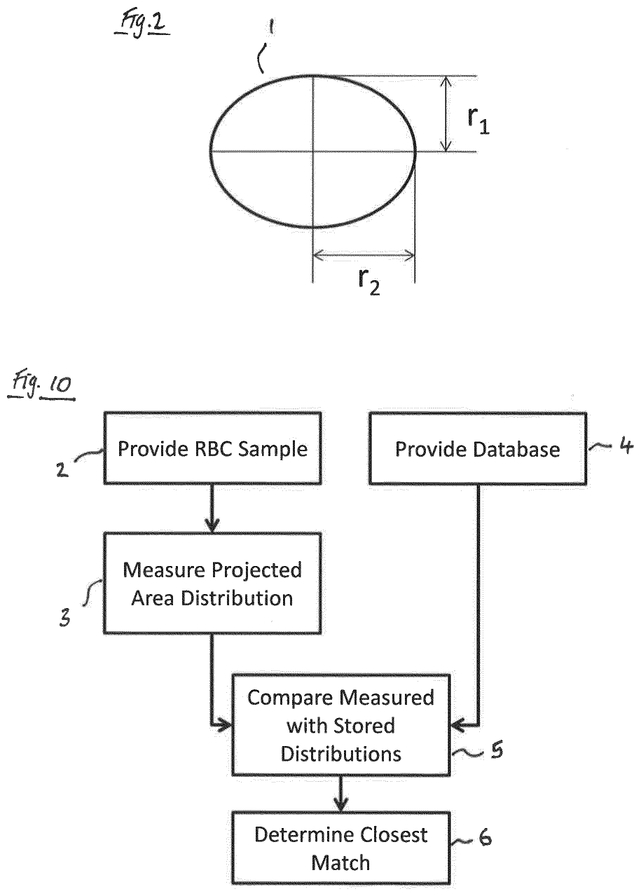

The projected cell areas were measured by image analysis, approximating the cell borders as an ellipse having major and minor radii r₁ and r₂, and the projected area being measured as π, r₁, r₂. FIG. 2 illustrates this measurement showing the ellipse 1 and the major and minor radii r₁ and r₂.

Returning to FIG. 1, eight characteristic peaks can be seen, each one centred approximately around the projected cell areas indicated. The distribution of projected areas is presented as a probability distribution, the area under the curve between any two projected areas being the probability of finding a cell within that area range.

The inventors were able to associate each of these peaks with a particular cell morphology, by visual inspection. The peaks corresponded to morphological forms as follows:
Peak 1: terminal echinocytosis
Peak 2: echinocytosis
Peak 3: stomato-RBC
Peak 4: stomato-biconcave
Peak 5: tense biconcave
Peak 6: common biconcave
Peak 7: wavy relaxed biconcave
Peak 8: relaxed biconcave

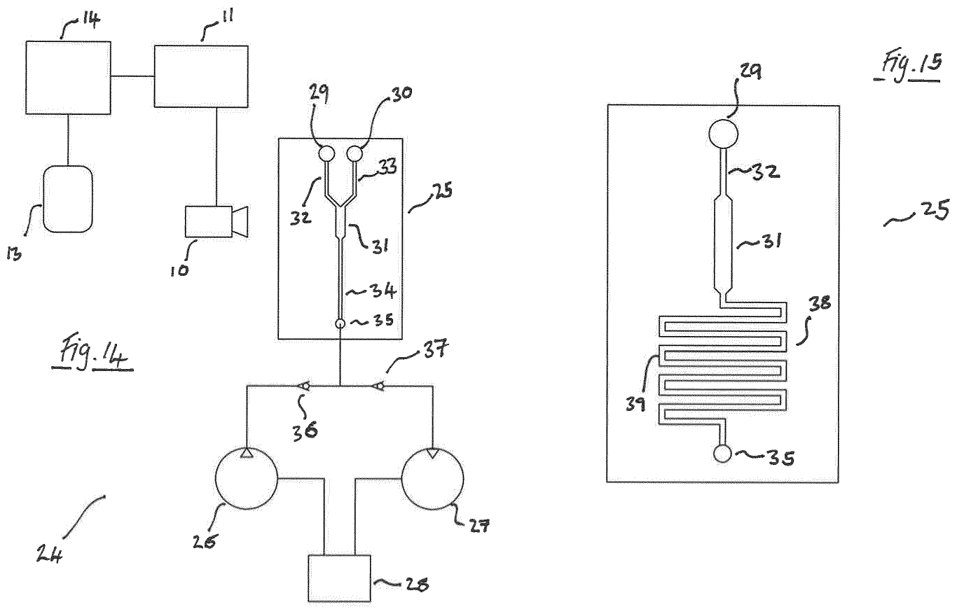

The morphological forms of these cells are illustrated in FIG. 13, showing terminal echinocytosis 16, echinocytosis 17, stomato-RBC 18, stomato-biconcave 19, tense biconcave 20, common biconcave 21, wavy relaxed biconcave 22 and relaxed biconcave 23 cells.

The inventors obtained blood samples from subjects that had been diagnosed with a range of rare anaemias, and performed the same analysis.

FIG. 3 shows the projected area distributions of RBCs from four subjects (identified as P005, P007, P053 and P059) diagnosed as having a mutation in the gene coding for the Band 3 anion exchanger protein (also known as anion exchanger 1 [AE1], band 3, or solute carrier family 4 member 1 [SLC4A1). It can be seen that, although there is some intra-subject variability, each of the morphological spectra (i.e. the area distributions) show very similar characteristic features.

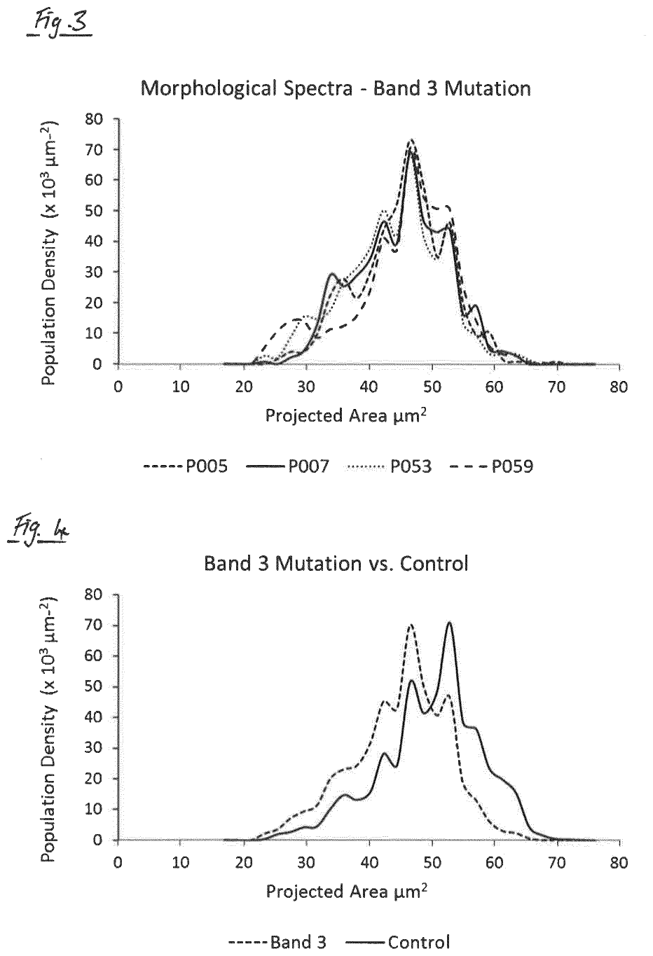

FIG. 4 shows the average projected area density distributions of RBCs for subjects having the Band 3 mutation vs. average plot for 11 healthy control subjects. The differences in the spectra are evident, for example, and most notably, the difference in relative sizes of peaks 5 and 6 (located at 46 μm² and 53 μm² respectively).

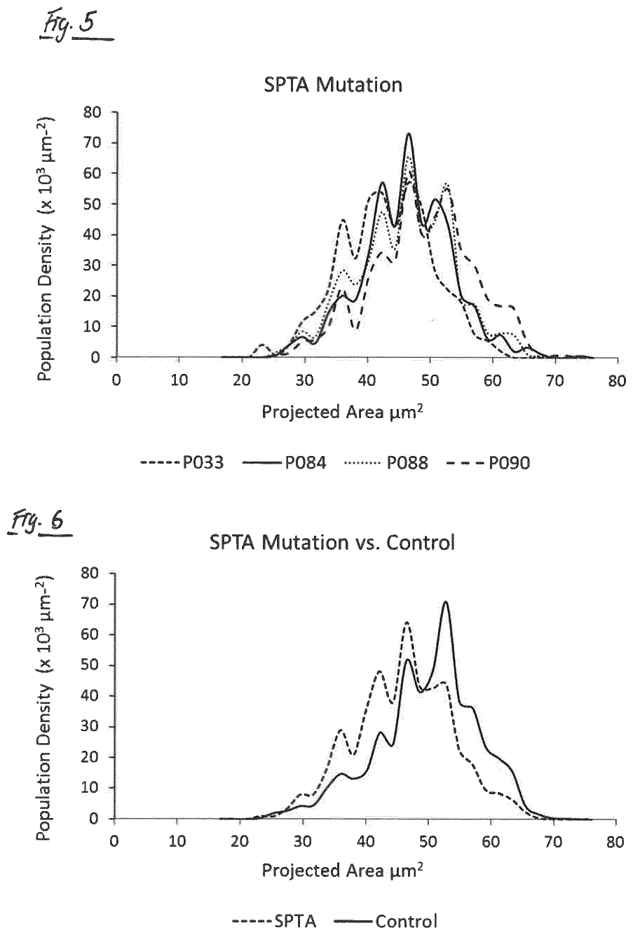

FIG. 5 shows the projected area distributions of RBCs from four subjects (identified as P033, P084, P088, and P090) diagnosed as having a mutation in the SPTA gene, coding for erythrocytic spectrin alpha chain protein, causing blood disorders including spherocytic haemolytic anemia (hereditary spherocytosis)). Again, although there is some intra-subject variability, each of the morphological spectra (i.e. the area distributions) show very similar characteristic features.

FIG. 6 shows the average projected area distributions of red blood cells for subjects having the SPTA mutation vs. healthy controls. Characteristic differences include a decrease in the relative abundance of cells having larger projected areas and a marked reduction in the proportion of cells in peak 6, located around 53 μm².

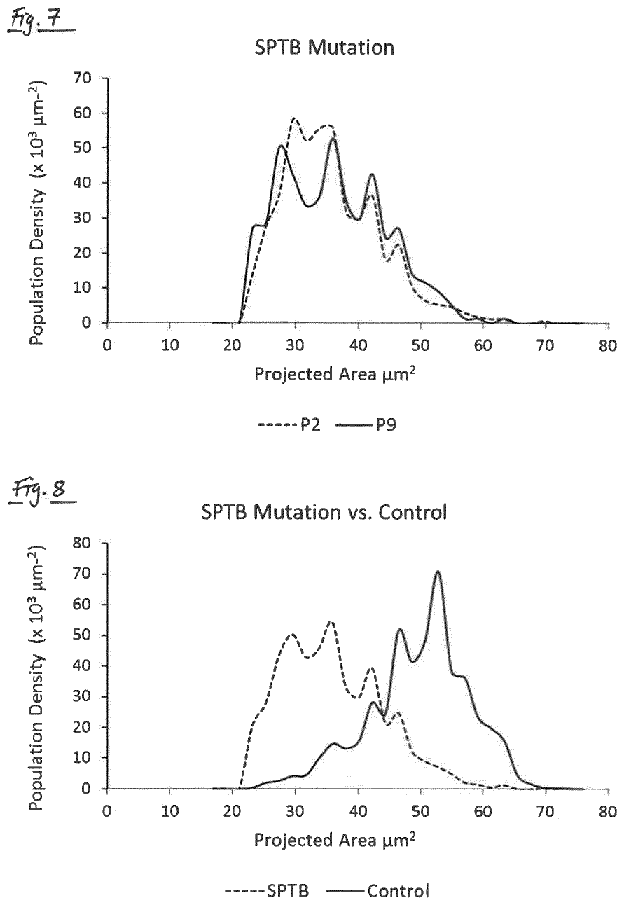

FIG. 7 shows the projected area distributions of RBCs from two subjects (identified as P2 and P9) diagnosed as having a mutation in the SPTB gene, coding for erythrocytic spectrin beta chain protein. Again, although some variability is present the two morphological spectra show similar characteristics.

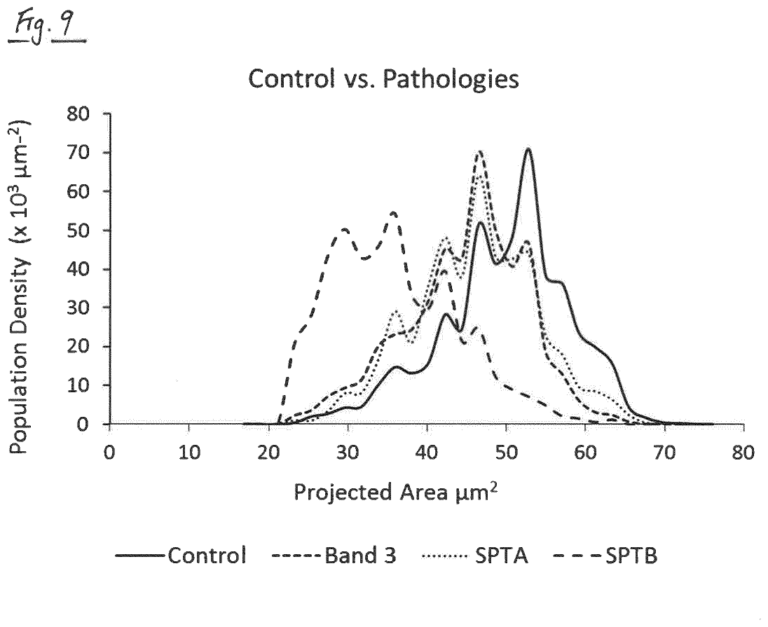

FIG. 8 shows the average projected area distributions of red blood cells for subjects having the SPTB mutation vs. healthy controls. Marked characteristic differences include a much higher presence of smaller projected area cells in the SPTB subjects compared to the controls.

FIG. 9 compares the projected area spectra for the three above pathologies vs. spectra from healthy control subjects.

The differences in these projected area spectrum therefor provide a method of differentiating between the different pathologies, or at least pre-screening subjects to select those who might benefit from a more detailed diagnostic study such as genetic sequencing.

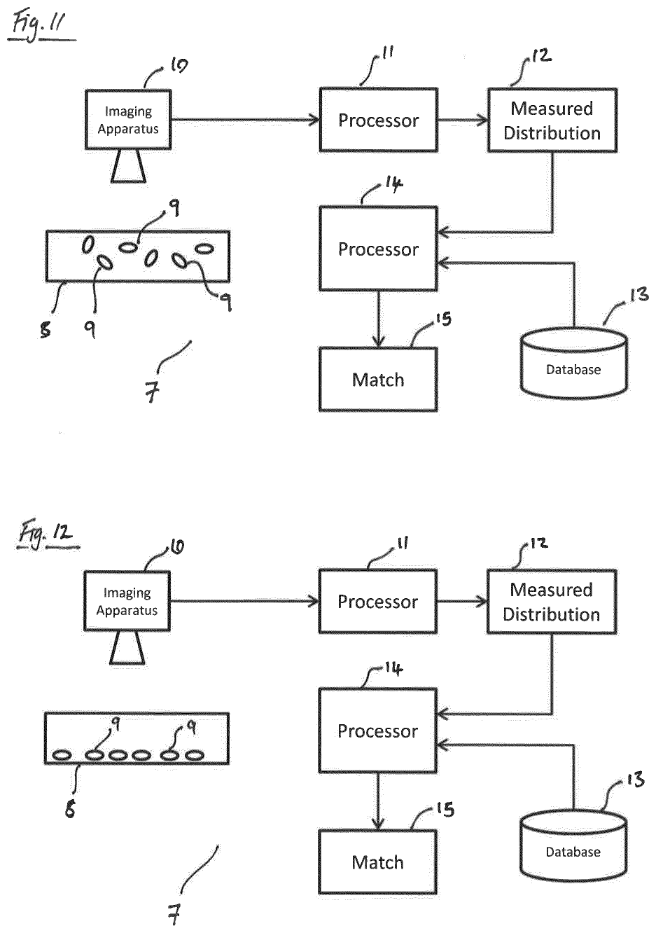

A variety of heuristics may be used to assign a putative pathology to a blood sample. FIG. 10 illustrates, as a flow diagram, a method for pre-screening subjects suspected of having an RBC disorder, or diagnosing such a disorder.

A sample of RBCs is provided 2, previously taken from a subject. A projected area distribution of the RBCs is measured 3. In order to take the measurement, the sample is preferably diluted. If sedimentation onto a surface is to be used to determine the maximum projected surface area, the sample is preferably diluted by approximately a factor of 1:1000, but this dilution could also by 1:5000, 1:2000, 1:500 or even 1:200.

The dilution medium should be selected so as not to change the morphological spectrum of the RBCs prior to measurement. The inventors have found that this may be achieved by using a medium that mimics blood plasma. In this regard, it is convenient to use a substitute such as the dilution medium described above. Alternative isotonic media should contain an albumin, preferably an albumin such as BSA (bovine serum albumin) or FCS (foetal calf serum). For example, PBS (phosphate buffered saline) or 0.9% NaCl solution each supplemented with 0.1% albumin, such as BSA would also be suitable.

Whilst the maximum projected area of each RBC may be conveniently measured by allowing the cells to sediment onto a surface, thereby orienting themselves with their largest surface area parallel to the plane of that surface, other methods of achieving this are also envisaged. For example, if the projected surface area of an RBC in free suspension is measured over time, as the cell is tumbling, a measure of the largest such projected area over time will give equivalent results. Similarly, if the RBCs are randomly oriented and not moving, then imaging the projected areas of the cells from multiple directions, and choosing the largest such projected area for each cell would also similarly produce equivalent results. Allowing the cells to sediment before imaging is, however, a simple and preferred method of achieving the projected surface area distribution. Such a method also aids in focussing the imaging apparatus, as all of the cells are essentially lying in the same plane.

It will also be appreciated that, although the present analysis is presented using a continuous distribution of projected cell areas, a similar analysis may be carried out by assigning each cell to a “bin” depending on its projected area. For example, by knowing the position of the peaks in the distribution, bins for each peak (and therefore each RBC morphology) may be constructed e.g. as follows:

| | Peak | Lower | Upper | |
|---|---|---|---|---|
| | Position | Bound | Bound | |
| Bin | (μm²⁾ | (μm²⁾ | (μm²) | Morphology |
| 1 | 25 | 20* | 27.5 | terminal echinocytosis |
| 2 | 30 | 27.5 | 33 | echinocytosis |
| 3 | 36 | 33 | 39 | stomato-RBC |
| 4 | 42 | 39 | 44 | stomato-biconcave |
| 5 | 46 | 44 | 49.5 | tense biconcave |
| 6 | 53 | 49.5 | 55 | common biconcave |
| 7 | 57 | 55 | 60 | wavy relaxed |
| | | | | biconcave |
| 8 | 63 | 60 | 65* | relaxed biconcave |

*The choice of these values may be chosen to exclude artefacts from imaging cell fragments, or platelets (for the lower bound), or larger cells such as white blood cells (for the upper bound).

A database 4 is also provided having representative RBC area distributions (either continuous or in bins, e.g. as above) taken from subjects with known pathologies, as well as healthy controls.

The measured projected area distribution of RBCs from the sample can then be compared 5 with those stored in the database to find a closest match 6.

Various methods of matching the measured distribution to the stored distributions will be evident to the skilled person. For example, among non-linear methods, a neural network may be trained to identify a measured peak as being most representative of a particular pathology.

Similarly, genetic algorithms could also be used to good effect. A linear approach could also be taken, such as the use of cross-correlation between measured and stored spectra, or a simple approach such as singular value decomposition may be used.

In order to demonstrate this, on the relatively small dataset obtained by the inventors thus far, a singular value decomposition analysis using a least squares approach was applied to the data.

Mean values for the projected area distributions for each of the pathologies and for control subjects were calculated. These data are those presented graphically in FIG. 9. These correspond to the database 4 of projected area number distributions.

Each of the patient and control samples was then compared against the database and a least squares difference calculated between each sample and the reference distributions. The least square difference (LSD) was calculated as follows:

$${LSD}_{j} = {\sum }_{1}^{n} {( {S}_{i} - {R}_{i , j} )}^{2}$$

Where:
LSDⱼ is the least square difference for reference distribution j;
Sᵢ is the population density of the sample at projected area i;

R_{i,j} is the population density of the reference distributions at projected area i for reference distribution j; and
where the continuous distribution is approximated by n data points.

The reference distribution having the lowest LSD gives an indication of the pathology represented by the sample. Table 1 shows the results of this analysis

TABLE 1
Determination of Closest Match

| | LSD compared to reference distributions | | | | RBC Pathology | | | % |
|---|---|---|---|---|---|---|---|---|
| Sample | Band 3 | SPTA | SPTB | Normal | Actual | Prediction | Correct | Correct |
| P005 | 327 | 840 | 15333 | 4972 | Band 3 | Band 3 | YES | 100% |
| P007 | 269 | 416 | 13563 | 4350 | Band 3 | Band 3 | YES | |
| P053 | 360 | 607 | 11415 | 5401 | Band 3 | Band 3 | YES | |
| P059 | 692 | 1141 | 15539 | 3119 | Band 3 | Band 3 | YES | |
| P033 | 2014 | 1800 | 9803 | 8907 | SPTA | SPTA | YES | 75% |
| P084 | 513 | 412 | 16201 | 4216 | SPTA | SPTA | YES | |
| P088 | 428 | 230 | 14219 | 2788 | SPTA | SPTA | YES | |
| P090 | 2124 | 1281 | 17602 | 714 | SPTA | Normal | NO | |
| P2 | 15794 | 15745 | 375 | 22758 | SPTB | SPTB | YES | 100% |
| P9 | 12056 | 12056 | 375 | 18939 | SPTB | SPTB | YES | |
| C006 | 12075 | 10131 | 26187 | 2800 | Normal | Normal | YES | 73% |
| C008 | 664 | 675 | 17358 | 2693 | Normal | Band 3 | NO | |
| C034 | 8064 | 6699 | 25335 | 819 | Normal | Normal | YES | |
| C054 | 3549 | 3108 | 22057 | 489 | Normal | Normal | YES | |
| C060 | 12751 | 11380 | 30132 | 2900 | Normal | Normal | YES | |
| C062 | 6268 | 5687 | 28023 | 1104 | Normal | Normal | YES | |
| C085 | 10394 | 8924 | 26628 | 1666 | Normal | Normal | YES | |
| C089 | 3829 | 3264 | 23637 | 519 | Normal | Normal | YES | |
| C091 | 518 | 305 | 12664 | 3592 | Normal | SPTA | NO | |
| C | 5640 | 4609 | 20359 | 549 | Normal | Normal | YES | |
| C2 | 725 | 641 | 12772 | 2810 | Normal | SPTA | NO | |

It can be seen that, even with the small amount of data used in this analysis, and with a simple least squares discriminator, the technique correctly identified the pathology associated with a sample for over 80% of all samples. It is expected that with more data, and a more sophisticated matching algorithm, better predictive power will ensue.

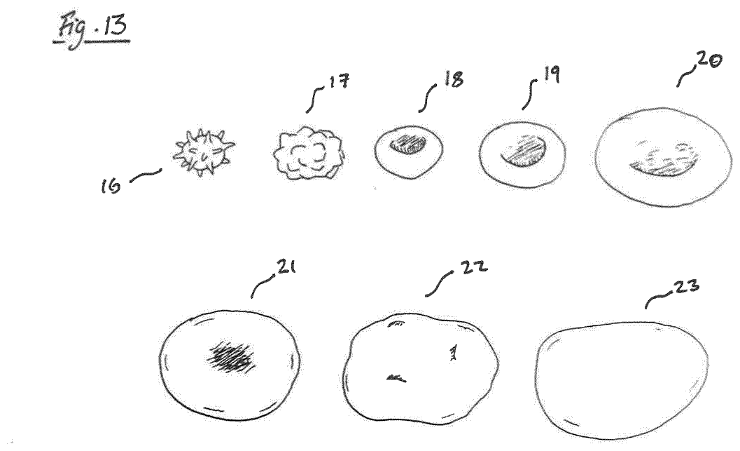

FIG. 11 illustrates, schematically, an embodiment of an apparatus for carrying out a method of pre-screening, diagnosis or patient monitoring as described herein, generally indicated by 7. The apparatus comprises a chamber 8 for accepting and holding a sample of red blood cells 9. In this embodiment, the cells 9 are suspended within the fluid in the chamber. They may be allowed to sediment, into a configuration illustrated schematically in FIG. 12 where they lie on a bottom surface of the chamber 8. Imaging apparatus 10 is also provided, e.g. in the form of a camera and microscope arrangement that can pass images to a processor 11 to produce a measured distribution 12. A database 13 is also provided, having projected cell area distributions from known pathologies and healthy subjects. The same 11 or a different processor 14 is also configured to compare the measured distribution 12 with those stored in the database 13, in order to find a closest match 15, and thereby indicate a putative assessment of the blood sample, e.g. by producing a diagnosis, or a pre-screening indicator.

Typically around 2000 RBCs are measured by automatic image analysis, but smaller numbers could also be counted, say 200, 500, or 1000 RBCs. As the field of view of most imaging systems is not able to image such numbers, the apparatus is preferably arranged to allow the field of view of the imaging apparatus and chamber to move relative to one another so that sufficient numbers of cells may be measured. In particularly preferred embodiments, a microfluidic cell may be employed, to allow samples to be moved into and out of an imaging chamber, which itself may be long enough to allow sufficient cells to sediment on its base such that they can be scanned with the imaging system.

Whilst this application has been described primarily in the context of identifying pathological conditions of red blood cells, the measurement of a morphological profile of a sample of red blood cells also has other applications.

Other diseases, such as diabetes, are known to affect the morphology of red blood cells in a subject, even though diabetes is not per se a pathology of RBCs. The techniques could also equally be used to identify disorders of RBC ion transport, and metabolic structure disorders of RBCs.

Furthermore, it is also known that as donated blood is stored, the morphological pattern of the red blood cells changes over time. The methods of measuring the morphological profile of a sample of red blood cells can therefore be applied to samples taken from stored, donated blood to assess potentially unwanted changes during storage.

The techniques described here could also be used to study osmotically induced red blood cell shrinkage or swelling and compensatory responses by the cells.

FIG. 14 illustrates apparatus for carrying out a method of RBC analysis as described herein, generally indicated by 24. The microfluidic test apparatus 24 comprises a microfluidic device 25, first and second pumps 26 and 27, respectively, and a controller 28 that operates the pumps 26, 27.

The microfluidic device 25 comprises a first reservoir 29 and a second reservoir 30 for receiving a first fluid and a second fluid, respectively, and a microfluidic test region 31 that may serve as the sedimentation chamber for analysis of the RBCs. A first microfluidic pathway 32 is provided between the first reservoir 29 and the microfluidic test region 31. A second microfluidic pathway 33 is provided between the second reservoir 30 and the microfluidic test region 31. In the microfluidic device 25 illustrated in FIG. 14, a further microfluidic pathway 34 is provided between the microfluidic test region 31 and a port 35.

The first pump 26 is connected to the port 34 via a valve 36. The first pump 26 and valve 36 are arranged to pump a priming fluid into the port 34 when operated. The first pump 26 may be a syringe pump, in which the syringe filled with the priming fluid. The priming fluid may contain a wetting agent to reduce air being trapped in the microfluidic device 25.

The second pump 27 is connected to the port 35 via a valve 37. The second pump 27 and valve 37 are arranged to apply suction at the port 35 when operated and draw fluid therefrom.

The valves 36 and 37 may take any suitable form, including a one-way valve, non-return valve, or an activated valve. In some embodiments the valves 36, 37 may be omitted.

The controller 28 is configured to control operation of the first and second pumps 26 and 27, and the valves 36, 376 where the valves are activated. The controller 28 may be any suitable device such as a microcontroller, embedded controller, programmable logic controller (PLC), microprocessor, portable computing device or computer and may include a control program. The controller 28 is configured to operate the first pump 26 to prime the microfluidic device 12. The first pump 14 preferably has a pump rate in the order of mL/second; this relatively high flow rate aids priming the microfluidic device 25 and reduces air entrapment. The controller 28 operates the first pump 26 to pump priming fluid into the microfluidic device 25 such that priming fluid enters the reservoirs 29, 30.

After priming, the first and second fluids are then added to the reservoirs 29 and 30, respectively. Where priming fluid has entered the reservoirs 29, 30, in some embodiments the priming fluid may be removed before the first and second fluids are added. The first fluid comprises a sample of red blood cells, which may be suitable diluted with a dilution medium as described above, typically at a dilution of approximately 1:1000. The second fluid is chosen according to the test requirements and may for example include a label and/or a stressor to the cells that cause a distinctive change in cells which may include cell lysis, aggregation, swelling, shrinkage, and/or shape change. In some embodiments, a series of second fluids may be added one by one to the second reservoir 30 as a test is performed, each second fluid having a different stressors, stressor concentration, and/or different labels.

If no stressor or other second reagent is required, the second reservoir 30 and its microfluidic passage 33 may be omitted.

The controller 28 is configured to operate the second pump 27 to draw a test volume of first fluid from the first reservoir 29 into the microfluidic test region 31. If used, a volume of second fluid will also be drawn from the second reservoir 30, according to the dimensions of the microfluidic pathways 32, 33. Since the second pump 27 applies suction to the port 35, pressure on the cells in the first fluid is limited. Using a pump to ‘push’ the first fluid through the microfluidic device 25 can result in higher pressure on the cells and cause cell ruptures, which may affect testing. It is preferred that the second pump 27 has a pump rate in the order of μL/second.

In the microfluidic device 25 shown in FIG. 14, the microfluidic test region 31 comprises a microfluidic channel into which the first and (if used) second fluids flow. Other forms of microfluidic test region 31 may be employed; for instance, the microfluidic test region 31 may comprise a microfluidic channel formed into a spiral. Forming the microfluidic test region 31 in a spiral may permit the imaging device 10 to capture fluid flow at several locations along the microfluidic test region 31 in a small area covered by a single image.

In order to perform an analysis as described herein a suitable protocol can include configuring the pump controller 28 to draw a sample of diluted RBCs into the microfluidic test region 31, to stop the flow to allow the RBCs to sediment in the microfluidic test region (i.e. to use it as a sedimentation chamber) and send a signal to the imaging system 10, 11 to cause it to capture an image of the sedimented cells. The pump controller can then restart the pump 27 to allow a further sample of diluted RBCs to be drawn into the microfluidic test region 31, and again stop the pump 27 and signal the imaging system 10, 11 to capture a further image of a second aliquot of diluted, sedimented RBCs. This can be repeated as often as required until a required number of cells have been imaged. The imaging system 10, 11 may be configured to send instructions to the pump controller 28 indicating whether sufficient images have been obtained.

It will be appreciated that the imaging apparatus 10 may be arranged to image the sedimented RBCs through either the top or bottom of the microfluidic device 25. Additionally, the microfluidic device 25 and imaging apparatus 10 may be configured such that the two are moved relative to each other after the cells have sedimented, to enable a larger field of view to be captured.

FIG. 15 illustrates, schematically, an alternative microfluidic device 25 that may be used as part of the apparatus. This device 25 differs from that illustrated in FIG. 14 in that a single reservoir 29 is provided, with a single microfluidic channel 32 connecting it to the microfluidic test region 31. This configuration is preferred where no second fluid (e.g. a stressor fluid or stain) is required in the methodology.

The device further comprises a microfluidic waste region 38 provided between the microfluidic test region 31 and the port 35. The microfluidic waste region 38 may comprise a circuitous microfluidic pathway 39. Whilst shown in two dimensions in the drawings for clarity it will be appreciated that the pathway 39 may be formed in three dimensions. The microfluidic waste region 38 defines a microfluidic volume commensurate with the test volume to prevent the test fluid from reaching the port 30. The microfluidic waste region 38 prevents the test fluid from leaving the microfluidic device 25, thereby avoiding cross-contamination that would result if some of the first or second fluids were to leave the microfluidic device 25 and then subsequently be pumped into another microfluidic device during the priming thereof. It will be appreciated that such a microfluidic waste region 28 may also be employed in an analogous fashion to the test device 25 of FIG. 13.

1. A method of measuring a morphological profile of a sample of red blood cells (RBCs) comprising the steps of:
(a) measuring a greatest projected area of each RBC in a population of said RBCs to create a projected area distribution of the RBCs; and
(b) identifying characteristic peak areas from said projected area distribution;
each relative peak area indicating the proportion of phenotypic cell types within the RBC sample.

2. The method according to claim 1, wherein said peak areas are the areas of those peaks located closest to the following projected areas:
Peak 1: 25 μm²
Peak 2: 30 μm²
Peak 3: 36 μm²
Peak 4: 42 μm²
Peak 5: 46 μm²
Peak 6: 53 μm²
Peak 7: 57 μm²
Peak 8: 63 μm²
and wherein each peak indicates the following RBC morphology:
Peak 1: terminal echinocytosis
Peak 2: echinocytosis
Peak 3: stomato-RBC
Peak 4: stomato-biconcave
Peak 5: tense biconcave
Peak 6: common biconcave
Peak 7: wavy relaxed biconcave
Peak 8: relaxed biconcave.

3. A method of pre-screening for a disorder affecting red blood cells (RBCs) in a subject comprising the steps of:
(a) providing a sample of red blood cells previously obtained from the subject;
(b) measuring a greatest projected area of each RBC in a population of said RBCs to create a projected area distribution of the RBCs;
(c) providing a database storing like projected area distributions of RBCs obtained from subjects including those with a range of such disorders;
(d) comparing said measured projected area distribution to those stored in said database to find a closest match, the potential RBC disorder being that associated with the matched distribution.

4. The method according to claim 3, wherein said disorder is a red blood cell disorder.

5. The method according to claim 4, wherein said disorder is an anaemia.

6. The method according to claim 5, wherein said disorder is selected from the group consisting of:
Hereditary Spherocytosis; and
Hereditary Xerocytosis.

7. A method of identifying a subject likely to have a mutation associated with a red blood cell (RBC) disorder comprising the steps of:
(a) providing a sample of red blood cells previously obtained from the subject;
(b) measuring a greatest projected area of each RBC in a population of said RBCs to create a projected area distribution of the RBCs;
(c) providing a database storing like projected area distributions of RBCs obtained from subjects including those with a range of such mutations;
(d) comparing said measured projected area distribution to those stored in said database to find a closest match, the potential mutation being that associated with the matched distribution.

8. The method according to claim 7, wherein said mutation is associated with a gene selected from the group consisting of:
ANK1 (coding for erythrocytic Ankyrin 1 protein);
SPTB (coding for erythrocytic spectrin beta chain protein);
SCL4A1 (coding for solute carrier family 4 member 1);
SPTA (coding for erythrocytic spectrin alpha chain protein); and
EPB42 (coding for erythrocyte membrane protein band 4.2).

9. The method according to claim 1, wherein the greatest projected area of each RBC is measured by:
(a) allowing said RBCs to sediment onto a surface; and
(b) measuring the projected surface area of each RBC in a direction substantially normal to the plane of said surface.

10. The method according to claim 1, where said sample of red blood cells is diluted before measurement.

11. The method according to claim 10, wherein said cells are diluted with a diluent containing an albumin.

12. The method according to claim 1, wherein said cells are exposed to a stressor before measurement.

13. The method according to claim 12, wherein said stressor is selected from the group consisting of:
osmotic stress;
pH stress;
temperature stress; and
mechanical stress.

14. The A method according to claim 1, wherein said projected area is measured by superimposing an ellipse over an image of each RBC, said ellipse having major and minor radii r₁ and r₂, and said projected area being measured as π, r₁, r₂.

15. The method according to claim 3, wherein said database of like projected area distributions contains averaged projected area distributions taken from a plurality of subjects.

16. An apparatus for carrying out the method of claim 1 comprising:
(a) a chamber for accepting a sample of RBCs;
(b) an imaging apparatus to image cells within said sedimentation chamber;
(c) a processor configured to measure the distribution of greatest projected areas of said cells;
(d) a database storing projected area distributions of RBCs obtained from subjects including those with a range of disorders affecting RBC morphology; and
(e) a processor configured to compare said measured distribution of projected areas to those stored in said database to find a closest match between said measured and stored projected area distributions.

17. The apparatus according to claim 16, wherein said chamber is a sedimentation chamber.

18. The apparatus according to claim 16, wherein said sedimentation chamber comprises a microfluidic chamber.
